(19) United States
(12) Patent Application Publication
(10) Pub. No.: US 2022/0312997 A1
(43) Pub. Date: Oct. 6, 2022

(54) DEVICES, SYSTEMS, AND METHODS FOR MONITORING FLUID INTAKE, HEALTHCARE GAMIFICATION, AND HEALTH PREDICTION

(71) Applicant: CME OFFSHORE sal, Beirut (LB)

(72) Inventors: Wissam Michel Youssef, Beirut (LB); Jad Mounir Trad, Beirut (LB); Bassel Adel Hassan, Beirut (LB); Elie Joseph Wakim, Beirut (LB); Ali Olleik, Beirut (LB)

(21) Appl. No.: 17/685,241

(22) Filed: Mar. 2, 2022

Related U.S. Application Data
(60) Provisional application No. 63/165,078, filed on Mar. 23, 2021.

(51) Int. Cl. A47G 23/16; G01F 15/06; A61B 5/00

(52) U.S. Cl. CPC A47G 23/16; G01F 15/068; A61B 5/4875

(57) ABSTRACT

Devices, systems, and methods are provided for monitoring water or other fluid consumption. For example, a fluid intake measurement device may provide highly accurate fluid consumption, which can be directly attached to a variety of fluid containers. The systems and methods herein also relate to the application of game psychology and mechanics to predict potential health issues and drive behavioral change for a healthier lifestyle and/or support motivational techniques for better healthcare conduct.

### RELATED APPLICATION DATA

The present application claims benefit of co-pending U.S. provisional application Ser. No. 63/165,078, filed Mar. 23, 2021, the entire disclosure of which is expressly incorporated by reference herein.

### TECHNICAL FIELD

The present application relates to devices, systems, and methods for monitoring water or other fluid consumption. More particularly, the present application relates to a fluid intake measurement device that provides highly accurate fluid consumption, which can be directly attached to a variety of fluid containers in an easy to attach fashion. The systems and methods herein also relate to the application of game psychology and mechanics to predict potential health issues and drive behavioral change for a healthier lifestyle and/or support motivational techniques for better healthcare conduct.

### BACKGROUND

With reduced water intake, bad sleeping patterns and minimal physical activities continuing to impact the health of people of all ages, the need to accurately measure and monitor water intake, sleeping patterns, and physical activities for a person to identify usage patterns and motivate people to stay hydrated, active, and sleep well is as important as ever. Measuring sleeping patterns and physical activities without the water intake may lead to the wrong medical assessment of a person. A person who exercises on a daily basis and follows a healthy sleeping time would require a different amount of water intake than a person who rarely exercises. That is why the combination of all three parameters (water intake, sleeping pattern and exercise time) may be a better indicator for a person's current status and a better representation to predict potential health issues in the future.

In addition, with the rising healthcare cost, healthcare payers (medical TPAs, insurers and self-funded groups) are shifting management to prevention of medical claims by promoting a healthier lifestyle that would ultimately lead to a reduced healthcare long-term cost. Healthcare payers would definitely need better assessment tools to predict the future cost of healthcare claims while trying to reduce them.

It is also important to have accurate measurements when communicating to health practitioners or signing up for new health insurance. In order to determine that one is meeting desirable hydration amounts and patterns, proper level of daily exercise, and sleep e.g., according to their body mass indicator, accurate measurements are highly important.

All existing healthcare gamification solutions depend on smart wearables to track a person's activities but without measuring or monitoring water intake. Traditional water intake monitoring methods typically depend on manually counting the number of cups consumed per day. However, this type of measurement provides a coarse reading of overall usage and is prone to errors resulting from human errors. For example, this measurement is often simply written down in notes and then filled manually into a smart app, and so entries are often missed.

In order to obtain more granular water intake measurement and to better understand and track water intake patterns, a variety of intake measuring devices have been created. However, the available metering devices all have several shortcomings.

For example, available water intake measurement devices (connected water bottles) often are designed to measure the amount of water in the remaining container. They typically comprise weight, Time-of-Flight, or capacitive sensors to measure the amount available in the container and deduce the difference that happened after drinking. While measuring the amount of liquid in a container may be effective in measuring water intake, such devices have several drawbacks. They highly depend on the specific designed container, which is critical for the measurement method used.

One problem with known solutions is that they highly depend on the position and orientation of the container. For example, a measurement method based on a weight sensor requires the container to be placed on a flat horizontal surface. A measurement method based on Time-of-Flight sensor does not work if the container is tilted upside down. A measurement method based on a capacitive sensor requires the container to stay straight during measurements.

Another problem with measuring the amount remaining in the container is that the user can discard the water from the container and this would be blind to the measurement instrument.

What is needed then is a device and system which can offer simplified attachment to different types of containers regardless of their size, form, and material, and can offer highly accurate reading for the amount consumed by the user.

What is also desired is to provide a device and system that monitor the amount of fluid consumed with respect to the time of the day and cannot get fooled by discarding fluid from the container.

What is additionally desired is a highly accurate measurement device and system with precise measurement of fluid exiting the container at the drinking location where communication is easily facilitated.

### SUMMARY

The present application is directed to devices, systems, and methods for monitoring water or other fluid consumption. More particularly, the present application is directed to a fluid intake measurement device that provides highly accurate fluid consumption, which can be directly attached to a variety of containers, e.g., in an easy to attach fashion. The systems and methods may also use the application of game psychology and mechanics to predict potential health issues and/or drive behavioral change for a healthier lifestyle and/or support motivational techniques for better healthcare conduct. The devices, systems, and methods may be young, fun and friendly-oriented with high interactive and engaging digital experiences with smart objects.

Therefore, in one aspect, systems and methods may be provided that can offer a software solution including one or more of an administration web application, a medical claims tracking system (CMS) and/or a mobile application that integrates with Health APIs like Apple Health and Fitbit API, and/or communicates with a smart fluid bottle through Bluetooth or other wireless communication protocols to measure a user's daily and weekly progress.

Optionally, the admin web application may be used to configure formulas for healthcare payer's members.

In one example, a formula or algorithm may be used that defines enabled key performance indicators (KPIs) such as steps walked, active calories burned, sleeping pattern, fluid consumed, etc. A given KPI of an insurance member (referred to as the mobile app user) is only tracked if the formula includes the KPI, and if the user gives access permissions to their data.

Each of the KPIs has an automatically calculated target given one or more of a member's age group, gender, and body mass index (BMI). The calculation of targets may be based on formulas widely adopted by health doctors and nutritionists.

For example, a formula can be applied for one or more policy/plan combinations.

In addition or alternatively, a formula may define how many points a user can be rewarded when they reach their target.

In another example, the mobile app user gives permissions to access his or her health data from an electronic health tracker device, like Apple Health, Garmin, Fitbit, and Huawei Health.

In still another example, the mobile app user always has visibility on the user's required targets and how many times they should reach them per week to get promised points.

Optionally, a marketplace containing a variety of physical and virtual rewards is available for the mobile app user to browse.

For example, the marketplace is configured from the web application by defining each reward's description, image, and cost in points.

When a user collects enough points to redeem a reward they like, they can redeem their points for it.

On redemption, the cost of the redeemed reward in points is deducted from the user's total points.

Optionally, the system may include a leaderboard, which may be available for users belonging to the same policy/plan to compete against each other. For example, if it is a monthly leaderboard, the rankings reset at the start of a new month. The name and image of the winning user may remain visible to all participants during the next month's competition, e.g., until the next winner is crowned.

Optionally, users may be smartly prompted by the app every once in a while, to update their weight and/or other information, e.g., so the app can intelligently track the impact of the program on their health.

Optionally, users have access to a point history that lists all the points acquired since they joined the game.

Optionally, a user can subscribe to notifications that will notify them when they acquire new points, motivate them to reach their closest-to-complete targets, let them know when the first rank in the leaderboard changes, and remind them to have enough sleep.

Optionally, users can buy a container using the app, and have the container shipped to their doors or other desired location.

Optionally, a user pairs a bottle to the app with a single click of ‘Pair.’ The app is able, from that moment, to know the time of each sip taken from the bottle.

Optionally, a user who is paired to a container can choose to be reminded to drink water and/or other fluids throughout the day through notifications.

In another example, a medical claim tracking system (CMS) may receive electronic as well as scanned medical claim files for every user and classifies them according to the International Classification of Decease and Current Procedural Terminology.

In another example, a machine learning algorithm learns from the medical claims set (CMS) and the user behavior (represented by health KPIs) to predict the health status of the user for the upcoming years.

Optionally, the mobile app pairs with any new health trackers to be seamless, regardless of the authorization requirements of each tracker. The mobile app doesn't have to differentiate between the different types of trackers.

Other aspects and features of the present invention and its particular features and advantages will become more apparent from consideration of the following drawings and accompanying detailed description.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIG. 1 shows an example of a fluid intake and monitoring device including a measurement device or sensor assembly attached to a fluid container.

FIG. 2 shows a cross-sectional view of the device shown in FIG. 1.

FIG. 3 shows an exploded view of an example of a measurement device/sensor assembly that may be included in the device of FIG. 1.

FIG. 4 shows an example of an attachment thread and fluid inlet that may be provided in the device of FIG. 1.

FIG. 5 shows an example of a mouth piece (water outlet) that may be provided on the device of FIG. 1.

FIG. 6 is a schematic of an exemplary system including a fluid container, such as the device of FIG. 1, a mobile electronic device, and a health tracker.

FIG. 7 is a schematic showing an exemplary network architecture of a system for healthcare gamification and health prediction, for implementing the devices, systems, and methods herein.

FIG. 8 shows another exemplary screenshot showing a user's progress towards reaching a daily or a weekly goal.

FIG. 9 shows an exemplary screenshot that may be presented on an electronic device using including a total unredeemed points of a mobile app user and the user's progress on a given day.

FIG. 10 shows an exemplary screenshot showing an example of a user's custom instructions that they need to follow to collect promised points.

FIG. 11 shows an exemplary screenshot of a marketplace where members can redeem their points for rewards they like.

FIG. 12 shows an exemplary screenshot of a leaderboard where members compete for the first rank on monthly basis.

FIG. 13 shows an exemplary screenshot of a point history where a user can view a list of their acquired points over time.

FIG. 14 shows an exemplary screenshot of a checkout screen when buying a smart water bottle from the app.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Referring now to the drawings, wherein like reference numerals designate corresponding structure throughout the views. The following examples are presented to further illustrate and explain the devices, systems, and methods herein, and should not be taken as limiting in any regard.

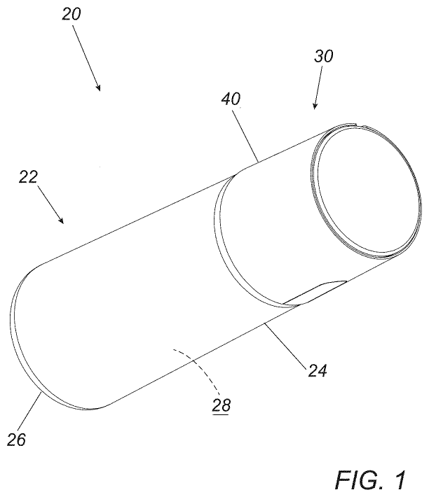

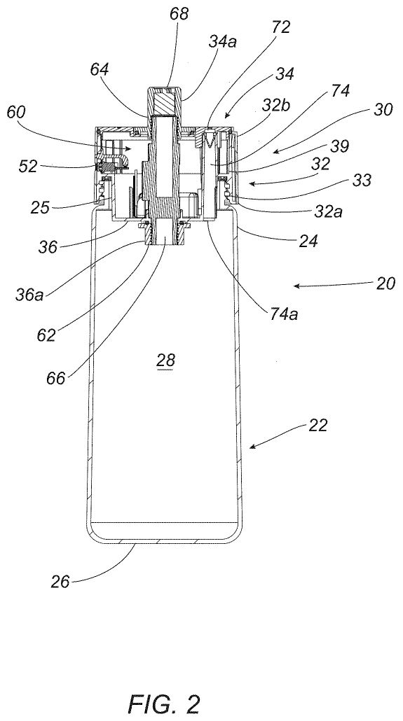

Turning now to FIGS. 1 and 2, an example of a fluid intake and monitoring device 20 is shown that includes a container 22 and a measurement device or sensor assembly 30. Generally, the container 22 includes an open upper end 24, a closed lower end 26, and an interior 28 for holding water or other fluid. As shown, the container 22 has a generally cylindrical cross-section between the ends 24, 26, although alternatively, the container may have other non-circular cross-sections and/or the cross-section may vary along the length of the container (not shown), as desired. Similar to conventional sports or water bottles, the container 20 may be formed from any desired material, e.g., metal such as stainless steel or aluminum, glass, plastic, and the like. As can be seen in FIG. 2, the upper end 24 may include a circular neck or extension 25 including one or more threads or other connectors for securing the sensor assembly 30 to the container 22, as described elsewhere herein.

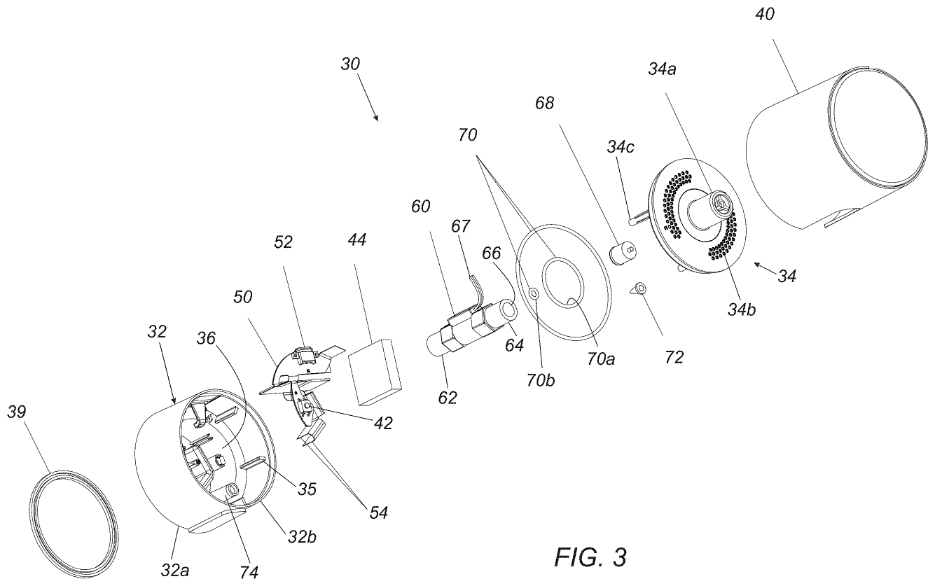

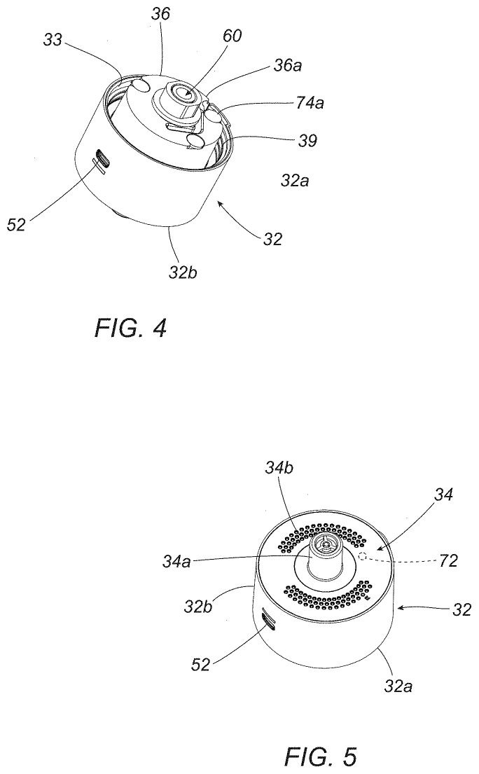

With additional reference to FIGS. 3-5, the sensor assembly 30 includes a housing 32 and a mouth piece 34 that contain mechanical and/or electronic components of the sensor assembly 30. The housing 32 may have a generally cylindrical or other shape, e.g., corresponding to the upper end 24 of the container 22, e.g., including a tubular sidewall 34 and a closed lower end 36. The housing 32 may include one or more connecters for securing the sensor assembly 30 to the container 22. For example, as shown in FIG. 4, the housing 32 may include one or more female threads 33 within the lower end 32a of the housing 32 that may be sized to slidably engage corresponding male threads 25 on the open end 24 of the container 22. Alternatively, it will be appreciated that other connectors may be used to removably secure the sensor assembly 30 to the container 22, e.g., such as snap-fit, interference fit, and/or other connectors (not shown).

Optionally, the sensor assembly 30 may include one or more seals, e.g., an O-ring or other annular seal 38 that may be secured adjacent to a lower end 32a of the housing 32, e.g., above the male threads 33, as best seen in FIG. 4, to provide a fluid-tight seal when the housing 32 is secured to the open end 24 of the container 22. The housing 32 may be removably connected to the neck 25 of the container 22, e.g., removable to facilitate filling the interior 26 with fluid, whereupon the housing 32 may be threaded back onto the neck 25 for use, with the seal 38 preventing fluid leakage.

Optionally, as shown in FIGS. 1 and 3, a cover 40 may also be provided that may be connected to the sensor assembly 30 and/or container 22, i.e., that is easily removable to allow water or other fluid within the container 22 to flow through the mouth piece 34 when a user drinks from the device 20. For example, a top surface of the cover 40 and the mouth piece 34 may include cooperating magnetic elements such that the cover 40 is magnetically drawn over the housing 32 until the magnetic elements engage to secure the cover 40. The magnetic force may be sufficiently strong to prevent the cover 40 from accidentally falling off, while also allowing a user to easily remove the cover 40 to drink the contents of the container 22. In addition or alternatively, other connectors may be provided, e.g., mating threads, detents, and the like (not shown) on a lower end of the cover 40 and on the housing 32 and/or container 22 (not shown) that may be engaged to secure the cover 40 while allowing easy removal.

With particular reference to FIGS. 2 and 3, the sensor assembly 30 may include several mechanical and/or electronic components secured within the housing 32, such as a controller or processor 42 and a battery or other power source 44, e.g., mounted on a PCB 50 that may be seated within the housing 32. As used herein, the term “processor” may include a single processor or a plurality of general or special-purpose processors that perform various functions of the detector device sensor assembly 30.

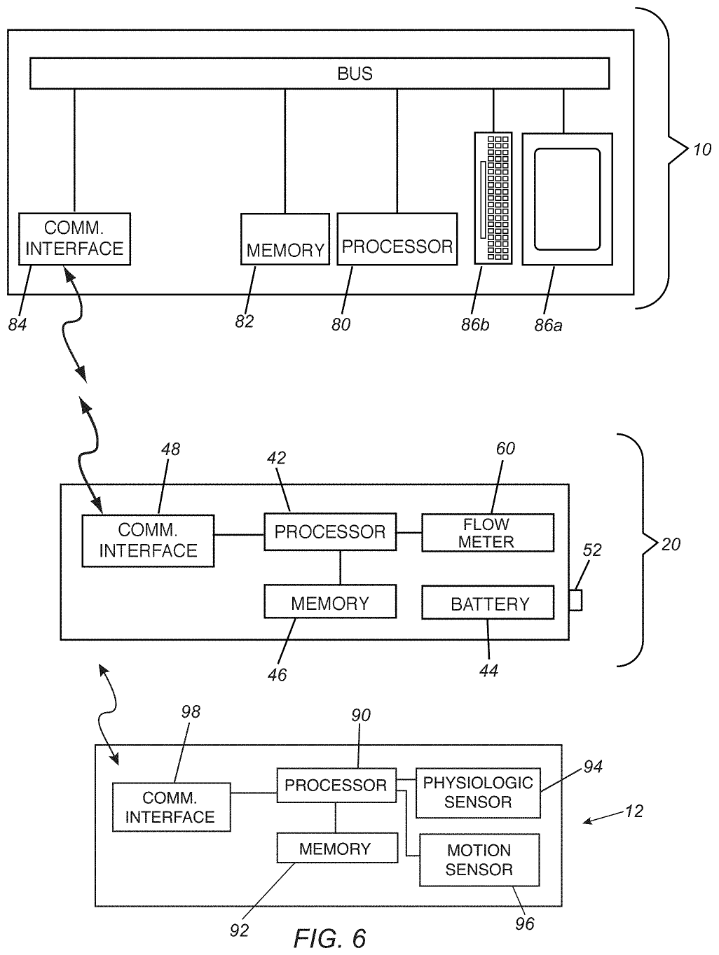

In addition, additional electronic components may also be mounted on the PCB, e.g., memory 46, a real-time clock 47, and/or a wireless communications interface 48 (see FIG. 6), e.g., a wireless transmitter and/or receiver configured to communicate via short-range radiofrequency signals, such as Bluetooth or other protocols. A charging port 52 may be provided on the housing 32, e.g., coupled to the battery 44 and/or PCB 50, such that a power cord (not shown) may be connected to the port 52 to charge the battery 44 and/or provide electrical power to components of the sensor assembly 30, as desired.

In addition, the sensor assembly 30 includes a fluid flow path extending from an inlet 36a in the lower end 36 of the housing 32a to an outlet nozzle 34a on the mouth piece 34 to allow a user to drink the contents of the container 22. The sensor assembly 30 also includes a flow meter or sensor 60 mounted in the fluid path to measure the volumetric flow rate of fluid passing through the flow path when a user drinks. For example, as shown in FIGS. 2 and 3, the flow meter 60 may include a tubular sleeve including a first end 62 that may be positioned in the inlet 36a and a second end 64 positioned adjacent a pressure valve 68 mounted within the nozzle 34a of the mouth piece 34.

The sensor assembly 30 also includes an annular seal 70 that is positioned under the mouth piece 34, as shown in FIGS. 2 and 3, that includes a central opening 70a sized to accommodate the second end 64 of the flow meter 60, and a peripheral opening 70b communicating with a vent passage 74 extending through the housing 32 to an opening 74a in the lower end 36 of the housing 32. The mouth piece 34 includes one or more vent openings, e.g., openings 34b arranged around the nozzle 34a. An air vent member 72 may be mounted within the vent passage 74 directly below the vent opening(s) 34b, e.g., as shown in FIG. 5.

With particular reference to FIG. 3, during assembly of the sensor assembly 30, the electronic components, e.g., including the processor 42, battery 44, memory 46, and interface 48 may be mounted to the PCB 50 using conventional methods and then the PCB 50 may be seated within the housing 32. The flow meter 60 may be inserted into the housing 32 and coupled to the processor 42, e.g., via leads 67 connected to the PCB 50, and then the mouth piece 34 may be assembled to the housing 32.

For example, the seal 70 and air vent member 72 may be positioned in place under the mouth piece 34, the pressure valve 68 inserted into the nozzle 34a of the mouth piece 34, and then the mouth piece may be permanently attached to the upper end 32b of the housing 32, e.g., by one or more of interference fit, one or more cooperating connectors, bonding with adhesive, fusing, and the like. For example, as can be seen in FIG. 3, the mouth piece 34 may include longitudinal tongues 34c that may slidably engage corresponding tabs 35 within the housing 32 to enclose the components to prevent rotation and/or other subsequent removal of the mouth piece 34.

With reference to FIGS. 2 and 4, the inlet 36a allows water or other fluid within the interior 28 of the container 22 to enter the lumen 66 of the flow meter 60 directly, where the fluid flow is measured by one or more sensors before the fluid exits the nozzle 34a.

Optionally, as can be seen in FIG. 3, a sensor 54, e.g., a capacitive water presence sensor, may be provided on the PCB 50 and/or otherwise mounted to the lower end 36 of the housing 32 to detect whether there is fluid in contact with the lower end 36, e.g., to confirm that the container 22 hasn't been emptied. The processor 42 may be coupled to the fluid presence sensor 54 to receive signals to confirm that fluid is in proper contact before measuring fluid consumption by the user.

For example, the processor 42 may be coupled to the flow meter 60, e.g., via leads 67, to process signals from the flow meter 60 to determine one or more parameters, e.g., flow rate, volume of fluid delivered, and the like, based on the signals from the flow meter 60. The processor 42 may also receive signals from the water presence sensor 54 such that, when the sensor 54 indicates there is no further fluid contact, the processor 42 will discontinue measuring fluid flow parameters, which may otherwise result in measuring more fluid than is actually being consumed by the user.

During use, the user may apply suction to the nozzle 34a of the mouth piece 34, with the pressure valve 68 preventing the flow path from opening until the user applies a threshold vacuum, thereby restricting fluid from exiting the container 22 without the application of enough suction force. During such suction, the air vent member 72 opens to deliver air into the interior 28 of the container 22 via the passage 74, e.g. to regulate pressure.

With particular reference to FIG. 2, when suction is applied to the nozzle 34a of the mouth piece 34 and the entire device 20 is tilted down, the pressure valve 68 may open and water (or other fluid) may flow through the flow meter 60 and along the flow path. To compensate the volume of consumed fluid, the air vent member 74 opens and air enters into the interior 28 of the container 22 to equalize pressure. Optionally, if the container 22 is sufficiently flexible to allow a user to squeeze the bottle, increased internal pressure from such squeezing may also open the pressure valve 68 and/or the air vent member 74, e.g., when the user squeezes the container 22 to take a sip.

Turning to FIG. 6, the device 20 may be part of a system 8 including an electronic device 10, e.g., a mobile electronic device such as a cellphone, tablet, and the like, that may communicate with the sensor assembly 30, e.g., using a software application that may be installed on the electronic device 10. Optionally, the system 8 may also include an electronic health tracker 12, such as a smart watch, GPS, device, and the like, that may also communicate with the electronic device 10 during use of the system 8.

The mobile electronic device 10 generally includes one or more processors (one processor 80 shown), memory 82, a communication interface 84, and one or more user interfaces 86, e.g., a display or other output device 86a, and a touch screen, keyboard, mouse, touch pad, and/or other input device 86b. The input device(s) 86b may facilitate a user controlling and/or otherwise communicating with the processor 80 or other components of the electronic device 10, as well as the sensing assembly 30 and/or health tracker 12, while the output device(s) 86a may allow information to be presented and/or manipulated in a desired manner, e.g., to present a series of menus, fields, pages, and/or other images, as described elsewhere herein. In one embodiment, the electronic device 10 may include a touch screen (not shown) that may act as a display 86a and as an input device 86b, allowing the user to scroll through menus or images, and/or select icons, e.g., by touching or otherwise selecting corresponding images on the touch screen, as described elsewhere herein.

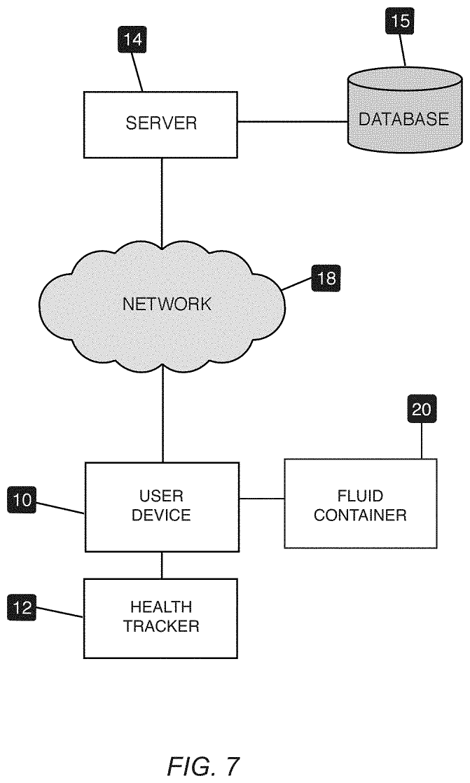

The communication interface 86 includes one or more transceivers, receivers, and/or transmitters, e.g., a short range transceiver for communicating with the sensing assembly 30 and/or health tracker 12 and/or a long range transceiver, e.g., to communicate with a server 14 and/or other electronic devices via a network 18, e.g., as shown in FIG. 7. In one example, the short range transceiver may communicate wirelessly using radiofrequency signals, such as Bluetooth or other protocols. Alternatively, a wired communication interface (not shown) may be provided for communicating with the sensing assembly 30 12, if desired, e.g., via port 52.

The long range communication interface may allow software and/or data to be transferred between the electronic device 10 and the server 14, and/or other external devices, networks, or information sources. For example, the long range communication interface may communicate via existing networks, e.g., telecommunications networks, the Internet, and the like, as represented by the network 18 shown in FIG. 7. Alternatively, the short range transceiver may be used to communicate with the server 14 and/or other remote devices via a local wireless network.

With continued reference to FIG. 6, the health tracker 12 generally includes one or more processors (e.g., one processor 90 shown), memory 92, one or more physiological sensors 94, a motion sensor 96, such as an accelerometer and/or gyroscope, and a communication interface 98. For example, the physiological sensor(s) 94 may include one or more sensors configured to measure various physiological parameters of a user wearing the health tracker 12, such as heart rate, breathing rate, oxygen saturation, and the like. The communication interface 98 may be a short range transceiver that communications with the mobile electronic device 10 via the communication interface 86, e.g., using radiofrequency signals, such as Bluetooth or other protocols. Optionally, the health tracker 12 may include additional components common to such devices, such as a battery or other power source, memory, and/or a GPS device (not shown).

The processor 80 of the electronic device 10 may receive signals from the sensor assembly 30, e.g., based on signals or data based on the flow meter 60, related to fluid consumption of a user of the fluid intake device 20, which may be incorporated into a health monitoring or other application installed on the electronic device 10, as described elsewhere herein. Optionally, the processor 80 of the electronic device 10 may receive signals from the health tracker 12, e.g., based on signals or data based on the physiological sensor(s) 94 and/or motion sensor 96. For example, an application on the electronic device 10 may correlate fluid intake information from the fluid intake device 20 with various actions of the user, e.g., identifying physical activities and/or conditions of the user based on data from the health tracker 12, as described elsewhere herein.

For example, at any time before use, a software application may be installed on the mobile electronic device 10, e.g., stored in memory 82, that allows the device 10 to communicate with the fluid intake device 20 and/or health tracker 12 and/or communicate information with a remote device, such as the server 14. The application may also include a series of menus to facilitate the user monitoring their fluid intake and/or participating in various activities, e.g., including competitions with other users of fluid intake devices similar to the device 20, claiming rewards based on identified accomplishments, and the like, as described elsewhere herein.

For example, the software application may be initially downloaded into memory 82 via the communication interface 86, e.g., from the server 14 and/or an independent application vendor's server. In addition, the application may be automatically upgraded at desired intervals, e.g., each time the fluid intake device 20 is coupled to the mobile electronic device 10, when the application is initially launched, when the network 18 is detected, and/or at periodic internals. During any such upgrades, firmware or software in the fluid intake device 20 itself may be upgraded, if desired, in addition to the application stored in the memory 82 of the mobile electronic device 10.

The following is an example of an algorithm that may be provided, e.g., in software applications installed on the electronic device 10 and/or sensor assembly 30 for monitoring fluid consumption by a user of the fluid intake device 20, e.g., for time synching without the clock 47.

For example, the sensor assembly 30 of the intake device 20 may include an algorithm in which the processor 42 that uses signals from the flow meter 60 to determine fluid flow and/or volume data that is stored in memory 46 as follows.
The processor 42 keeps track of the cumulative number of milliliters sipped through the mouthpiece 34 as long as the algorithm is not reset.
The processor 42 starts grouping sips taken by number of one-minute cycles that have passed since last sync between the sensor assembly 30 and the electronic device 10.
Each sip taken is saved in memory 46 with two (2) essential values, i.e., the number of one-minute cycles that have passed since that last sync between the sensor assembly 30 and the application (referred to herein as “elapsedMinutes”), and the cumulative number of milliliters recorded after the sip was taken (referred to herein as “cumulativeAfterSip”).

The application on the electronic device 10 may include an algorithm that operates as follows:
When a sensor assembly 30 from a fluid intake device 10 is paired to the electronic device 10, e.g., via the communications interfaces 48, 84, the application asks the sensor assembly 30 to restart counting sips, and saves the current time from the clock 47 in memory 46 (referred to herein as “lastSyncTime”).
The application also saves the cumulative number of milliliters read from and stored by the sensor assembly 30 since the last sync (referred to herein as “totalCumulativeMilliliters”).
Every time the application is required to read from the sensor assembly 30, the application will start by reading each sip saved in memory 46 from the fluid intake device 20.
For each sip, the application adds the “elapsedMinutes” to the “lastSyncTime” to discover the time at which this sip was taken. The number of milliliters is calculated based on the following formula:
   If first sip in storage: “cumulativeAfterSip”—“totalCumulativeMilliliters”
   Otherwise: “cumulativeAfterSip”—“cumulativeAfterSip” from previous sip.
The application may read the cumulative number of milliliters to find if there are extra milliliters drank in addition to the stored sips. This might happen if the one-minute cycle has not ended.
The application will then ask the sensor assembly 30 to restart counting sips and will update both “lastSyncTime” to now and “totalCumulativeMilliliters” the latest read.

Optionally, the application may detect a user's activity type using data from the motion sensor 96 of the health tracker 12. For example, machine learning may be used to learn from the data to identify patterns and provide predictions on the type of activity. For example, the application may detect if the data reflects toothbrushing activity or anything else. This is mainly a binary classification problem. The application may use discrete signal processing to process the data, e.g., to transform the data from its time domain to the frequency domain. Such processing may provide better results for multi-dimension signal classification problems. For example, it may be desirable to create discrete signals from the stream of points using a certain window of time, stride, and sampling rate, apply on it some signal transformation techniques and feed that to an ML classification algorithm.

Turning to FIG. 8, an exemplary screenshot is shown that may be presented on the display 86a of the electronic device 10 during use of the fluid intake device 20. As shown, the application installed on the electronic device 10 may track the user's water/fluid consumption and information regarding their consumption may be presented on the display 86a, e.g., to assist the user in maintaining target fluid consumption, e.g., based on daily, weekly, and/or other desired time periods. For example, the electronic device 10 may present fluid intake information, such as total water consumption (not shown) and/or percentage of target intake (e.g., 11%, shown). Optionally, if the user also uses the health tracker 12, additional information regarding the user's activities, such as steps taken, sleep time, training information, and the like, may also be presented to assist the user in maintaining targets for such activities.

Optionally, the application may include a reward program to help incentivize the user to reach target goals for water consumption and/or other activities. For example, as shown in FIG. 7, a server 14 may be maintained by an operator of the reward program to communicate with users of intake devices and systems, e.g., via network 16. The network 16 may include a private or public network, including a wide area network (“WAN”), a local area network (“LAN”), an intranet, a wireless network, a short messaging service (“SMS”), or a telephony network. For example, any such network may incorporate several different types of networks including a WAN, a LAN, and/or a wireless network. One such network including multiple different types of networks is the Internet.

The server 14 may include one or more computer systems, e.g., servers, communicating with one or more databases (one database 16 shown for simplicity), e.g., including one or more processors, memory and/or storage devices, and communication interfaces for communicating via the network 14, e.g., with users 10-n and/or other parties involved in the methods performed by the system. The server 14 may include one or more hardware-based components and/or software-based modules for performing the various functions related to the methods performed, as described elsewhere herein. Although only one server 14 is shown, it will be appreciated that multiple servers (not shown) may be provided at the same or different locations that operate cooperatively to perform the functions described herein.

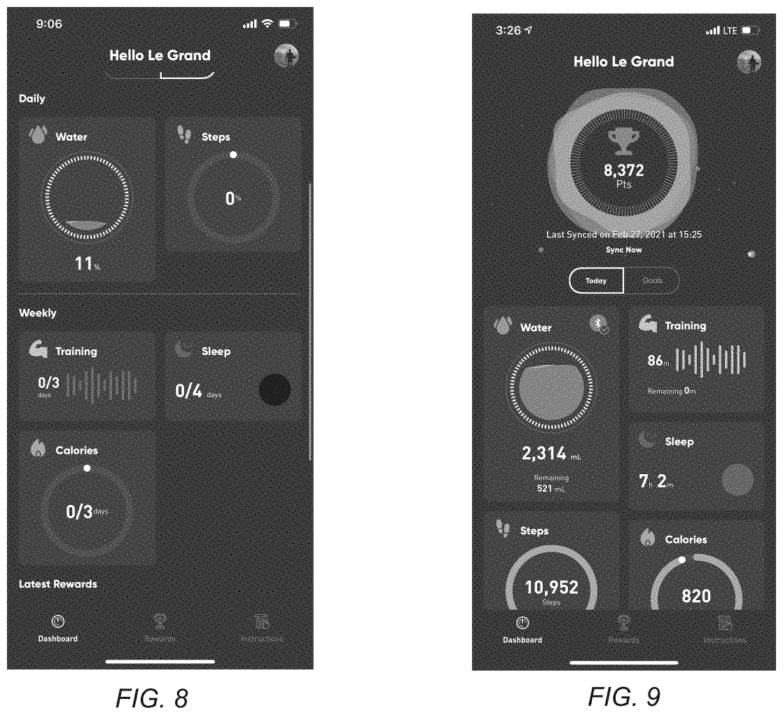

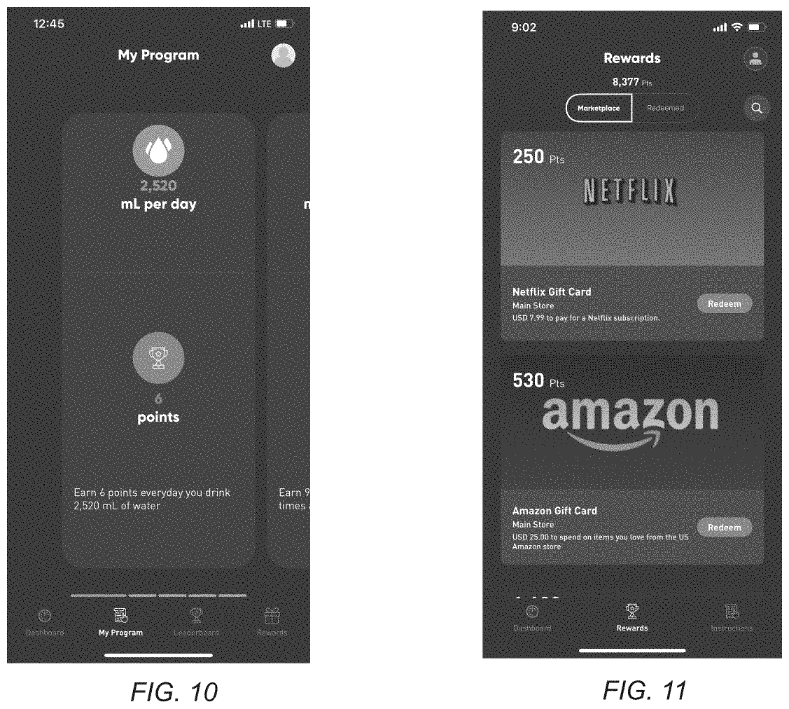

Turning to FIG. 9, one example of a program that may be offered to users is a points-based program that allows users to accumulate points for accomplishing target goals, e.g., meeting daily fluid consumption targets and/or performing target activities, such as exercise, sleep, and the like. As shown in FIG. 9, an exemplary screenshot shows a user having unredeemed reward points (e.g., 8,372), in addition to presenting water consumption and activity information. The application may allow the user to spend the points for various rewards offered by the program. For example, FIG. 10 shows an example of a user's custom instructions that they need to follow to collect promised points, e.g., “earn 6 points every day you drink 2520 mL of water” and may include their current status towards the goal required. FIG. 11 shows an example of offers that may be presented to a user so they can spend their reward points to purchase various goods or services. When the user selects a reward, the application may communicate the request from the electronic device 10 to the server 14, which may process and provide the reward. The server 14 may then deduct the points from the user's total and maintain the current balance, e.g., in the database along with other user information.

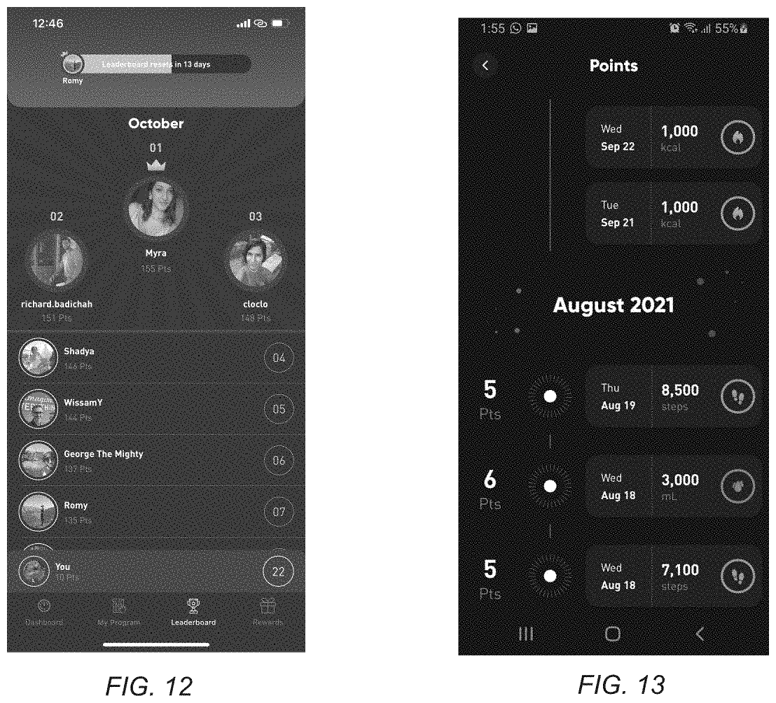

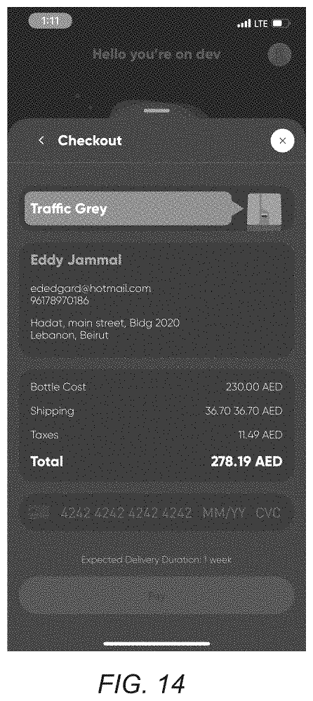

Turning to FIG. 12, an operator of the rewards program may also establish competitions and/or other activities between users of the fluid intake devices and systems. Such competitions may include generating leaderboards where users can compete for ranking, e.g., to create further incentives and/or rewards to users. For example, as shown, members may be ranked on monthly basis, e.g., with users' positions in the rankings changing over the course of a month (or alternatively other desired time period, e.g., weekly) based on their success at achieving targets. In the example shown, a user with the top rank may be presented prominently, e.g., at the top of a list of users along with additional information, such as their user name, total points (e.g., month-to-date). Such competitions may also enhance user's compliance with target goals, which may increase the chance of consuming proper amounts of water/fluids and/or completing activities that may enhance their overall health.

Optionally, as described elsewhere herein, users' information regarding their fluid consumption and/or activities may be shared with authorized parties, e.g., the users' healthcare providers, health insurance providers, and the like, e.g., to guide such providers in providing care to the users.

In describing representative examples, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for monitoring fluid consumption by a user, comprising:
a fluid container comprising an open upper end, a closed lower end, and an interior for holding water or other fluid; and
a sensor assembly comprising:
a housing connectable to the upper end of the fluid container such that a flow path communicates between the interior and a mouth piece on the housing;
a flow meter in the flow path configured;
a processor coupled to the flow meter for identifying when the user sips fluid via the mouth piece causing fluid to flow from the interior through the flow path to an outlet in the mouth piece, the processor configured to process signals from the flow meter to determine flow information for each identified sip; and
memory coupled to the processor for storing the flow information for each of the identified sips.

2. The device of claim 1, further comprising a pressure valve within the flow path for preventing fluid within the interior from traveling through the flow path and out the mouth piece unless a predetermined threshold suction is applied by the user when the user takes a sip.

3. The device of claim 2, wherein the pressure valve is configured to prevent fluid from escaping through the mouth piece unless the lower end of the container is higher than the upper end of the container.

4. The device of claim 1, further comprising an air vent passage in the housing communicating between the interior of the container and the mouth piece, and an air vent member configured to open when suction is applied to the mouth piece to deliver air into interior to equalize pressure within the interior.

5. The device of claim 1, further comprising a fluid presence sensor coupled to the processor configured to confirm fluid is present within the interior before identifying a sip and determining flow information.

6. The device of claim 1, wherein the processor is configured to determine a volume of fluid consumed by the user during each identified sip.

7. The device of claim 6, wherein the sensor assembly further comprises a clock, and wherein the processor is configured to store a time stamp from the clock with the volume of fluid determined for each identified sip.

8. The device of claim 6, wherein the processor is configured to determine the volume of fluid consumed in one-minute cycles.

9. The device of claim 1, further comprising a communications interface for transmitting data related to identified sips to another electronic device.

10. A device for monitoring fluid consumption by a user, comprising:
a fluid container comprising an open upper end, a closed lower end, and an interior for holding water or other fluid; and
a sensor assembly comprising:
a housing connectable to the upper end of the fluid container such that a flow path communicates between the interior and a mouth piece on the housing;
a flow meter in the flow path configured;
a processor coupled to the flow meter for identifying when the user sips fluid via the mouth piece causing fluid to flow from the interior through the flow path to an outlet in the mouth piece, the processor configured to process signals from the flow meter to determine flow information for each identified sip;
a fluid presence sensor mounted adjacent the upper end of the container and coupled to the processor, the processor configured to analyze data from the fluid presence sensor to confirm fluid is present within the interior before identifying a sip and determining flow information and
memory coupled to the processor for storing the flow information for each of the identified sips.

11. The device of claim 10, wherein the fluid presence sensor comprises a capacitive sensor mounted to the housing adjacent the upper end of the container, the processor configured to detect whether there is fluid in contact with the capacitive sensor to confirm that the container hasn't been emptied before determining flow information.

12. A system for monitoring fluid consumption by a user, comprising:
a fluid container and sensor assembly according to claim 1; and
an electronic device comprising:
a communications interface; and
an electronic device processor coupled to the communications interface for communicating with the sensor assembly to receive the flow information.

13. The system of claim 12, wherein the electronic device processor is configured to process the flow information received from the sensor assembly to determine cumulative fluid consumption of the user.

14. The system of claim 13, wherein the electronic device processor is configured to instruct the sensor assembly to reset flow information stored in the memory after receiving the flow information.

15. The system of claim 12, wherein the electronic device comprises a display coupled to the electronic device processor for presenting the flow information.

16. The system of claim 12, further comprising:
a health tracker device comprising a communications interface for communicating with the electronic device and one or more sensors configured to acquire data related to activities of the user; and
wherein the electronic device processor is configured to process signals from the one or more sensors to identify activities of the user and correlate the activities to the flow information.

17. The system of claim 16, wherein the one or more sensors comprise one or both of a physiological sensor and a motion sensor.

18. The system of claim 16, wherein the processor is configured to identify when the user is consuming insufficient fluid based on the identified activities and present notifications on the display advising the user to consume additional fluid.

19. The system of claim 15, wherein the processor is configured to present information on the display related to a reward system based on the user's fluid consumption.

20. The system of claim 19, wherein the processor is configured to present information regarding one or more of points available and points acquired in the reward system based on achievement of fluid consumption goals by the user.

21. The system of claim 15, wherein the communication interface of the electronic device is configured to communicate with a remote server, the processor configured to receive information regarding other users of fluid containers and present competitive information comparing fluid consumption of the user to the other users.

22. The system of claim 21, wherein the competitive information comprises presenting a ranking of the user based on their fluid consumption to fluid consumption of other users.

23. A method for monitoring fluid consumption by a user, comprising:
providing a fluid container comprising an open upper end, a closed lower end, and an interior for holding water or other fluid; and a sensor assembly connected to the upper end of the fluid container such that a flow path communicates between the interior and a mouth piece on the sensor assembly;
identifying, by a processor of the sensor assembly coupled to a flow meter in the flow path, when the user sips fluid via the mouth piece causing fluid to flow from the interior through the flow path to an outlet in the mouth piece; and
determining, by the processor based on signals from the flow meter, flow information for each identified sip.

24-25. (canceled)

26. The system of claim 16, wherein the electronic device processor is configured to correlate identified activities and the flow information and present information to the user on a display of the electronic device.
